(19) United States
(12) Patent Application Publication
(10) Pub. No.: US 2024/0001021 A1
(43) Pub. Date: Jan. 4, 2024

(54) MODIFIED YANKAUER SUCTION TIP AND TUBING ASPIRATOR FOR IMPROVED ASPIRATION DURING DENTAL OR SURGICAL PROCEDURES

(71) Applicant: JAMES STUART MELVIN, Washington, DC (US)

(72) Inventor: JAMES STUART MELVIN, Washington, DC (US)

(21) Appl. No.: 17/856,071

(22) Filed: Jul. 1, 2022

(51) Int. Cl. A61M 1/00; A61B 1/00

(52) U.S. Cl. CPC A61M 1/86; A61B 1/00094

(57) ABSTRACT

A suction tip and tubing aspirator is disclosed and comprises a first lumen having a first open end adapted to be operatively connected to a source of suction, and a second open end adapted to be inserted into a site from which tissue or debris is to be aspirationally removed, a second lumen integrally connected to the first lumen, and a stylus movably disposed within the second lumen between retracted and extended positions such that when the stylus is moved from the retracted position to the extended position, a distal end portion of the stylus will protrude outwardly from the second end of the first lumen so as to remove any tissue or debris which has clogged, blocked, or occluded the second open end of said first lumen.

### FIELD OF THE INVENTION

The present invention relates generally to the field of dental or surgical devices or implements, and more particularly to a modified or new and improved Yankauer suction tip and tubing aspirator which will easily and quickly unclog or unblock Yankauer suction tip and tubing aspirators should they become clogged, blocked, or otherwise occluded.

### BACKGROUND OF THE INVENTION

Aspiration devices are commonly utilized during various dental or surgical procedures. More particularly, Yankauer suction tip and tubing aspirators are commonly utilized during various dental, surgical, and anesthesia procedures. However, these devices have been known to become easily clogged, blocked, or occluded with bits of tissue or other debris generated during the aforenoted procedures. In order to prevent such clogging, blocking, or occlusion from occurring, some devices have utilized sponges, screens, filters, or the like. Still yet further, some devices have employed tips having variable-sized nozzles so as to effectively control the size of the suction orifice as may be desired or required. Nevertheless, even these modified aspirator tips have still experienced clogging, blocking, or occlusion, especially during those procedures where substantially large quantities of tissue, or other matter or debris, are to be removed by suction techniques. When such clogging, blocking, or occlusion occurs, precious time and effort is required to unclog the suction tip, and therefore such time effectively interrupts the primary procedure being done, which, of course, requires additional overall time to be expended in order to properly conduct and complete the primary procedure from start to finish. Alternatively, multiple suction aspirators have been utilized so as to effectively change or switch out the original clogged, blocked, or occluded suction aspirator with a new, fresh, second suction aspirator, however, this procedural mode obviously requires additional staff personnel to unclog or unblock the original suction aspirator so as to ready the same to be switched or changed with the second suction aspirator should that second suction aspirator likewise become clogged, blocked, or occluded. Examples of Yankauer or similar suction tip and tubing aspirators are disclosed within U.S. Pat. No. 8,834,407 which issued to Greeson, Jr. et al. on Sep. 16, 2014; U.S. Pat. No. 7,625,207 which issued to Hershey et al. on Dec. 1, 2009; U.S. Pat. No. 4,468,217 which issued to Kuzmick et al. on Aug. 28, 1984; U.S. Design Pat. No. 264,246 which issued to Ekbladh et al. on May 4, 1982; U.S. Pat. No. 4,204,328 which issued to Kutner on May 27, 1980; and U.S. Pat. No. 3,169,528 which issued to Knox III et al. on Feb. 16, 1965.

A need therefore exists in the art for a new and improved Yankauer suction tip and tubing aspirator. An additional need exists in the art for a new and improved Yankauer suction tip and tubing aspirator that can be utilized during various dental and/or surgical procedures. Another need exists in the art for a new and improved Yankauer suction tip and tubing aspirator wherein, when the aspirator tip becomes clogged, blocked, or occluded with bits of tissue or other debris generated during the aforenoted procedures, the new and improved Yankauer suction tip and tubing aspirator can easily and quickly unclog or unblock the clogged, blocked, or occluded aspirator. Still another need exists in the art for a new and improved Yankauer suction tip and tubing aspirator wherein, when the aspirator tip becomes clogged, blocked, or occluded with bits of tissue or other debris generated during the aforenoted procedures, the new and improved Yankauer suction tip and tubing aspirator can easily and quickly unclog or unblock the clogged, blocked, or occluded aspirator by quickly discharging any bits of tissue or other debris clogging, blocking, or occluding the aspirator tip.

Yet another need exists in the art for a new and improved Yankauer suction tip and tubing aspirator wherein, when the aspirator tip becomes clogged, blocked, or occluded with bits of tissue or other debris generated during the aforenoted procedures, the new and improved Yankauer suction tip and tubing aspirator can easily and quickly unclog or unblock the clogged, blocked, or occluded aspirator by quickly discharging any bits of tissue or other debris clogging, blocking, or occluding the aspirator tip without expending precious time, away from the primary procedure being performed, which would otherwise ordinarily need to be expended. A further need exists in the art for a new and improved Yankauer suction tip and tubing aspirator wherein, when the aspirator tip becomes clogged, blocked, or occluded with bits of tissue or other debris generated during the aforenoted procedures, the new and improved Yankauer suction tip and tubing aspirator can easily and quickly unclog or unblock the clogged, blocked, or occluded aspirator by quickly discharging any bits of tissue or other debris clogging, blocking, or occluding the aspirator tip without the need to switch or change to a new or fresh alternative or second aspirator.

### OVERALL OBJECTIVES OF THE PRESENT INVENTION

An overall objective of the present invention is to provide a new and improved Yankauer suction tip and tubing aspirator. An additional overall objective of the present invention is to provide a new and improved Yankauer suction tip and tubing aspirator that can be utilized during various dental and/or surgical procedures. Another overall objective of the present invention is to provide a new and improved Yankauer suction tip and tubing aspirator wherein, when the aspirator tip becomes clogged, blocked, or occluded with bits of tissue or other debris generated during the aforenoted procedures, the new and improved Yankauer suction tip and tubing aspirator can easily and quickly unclog or unblock the clogged, blocked, or occluded aspirator. Still another overall objective of the present invention is to provide a new and improved Yankauer suction tip and tubing aspirator wherein, when the aspirator tip becomes clogged, blocked, or occluded with bits of tissue or other debris generated during the aforenoted procedures, the new and improved Yankauer suction tip and tubing aspirator can easily and quickly unclog or unblock the clogged, blocked, or occluded aspirator by quickly discharging any bits of tissue or other debris clogging, blocking, or occluding the aspirator tip.

Yet another overall objective of the present invention is to provide a new and improved Yankauer suction tip and tubing aspirator wherein, when the aspirator tip becomes clogged, blocked, or occluded with bits of tissue or other debris generated during the aforenoted procedures, the new and improved Yankauer suction tip and tubing aspirator can easily and quickly unclog or unblock the clogged, blocked, or occluded aspirator by quickly discharging any bits of tissue or other debris clogging, blocking, or occluding the aspirator tip without expending precious time, away from the primary procedure being performed, which would otherwise ordinarily need to be expended. A further overall objective of the present invention is to provide a new and improved Yankauer suction tip and tubing aspirator wherein, when the aspirator tip becomes clogged, blocked, or occluded with bits of tissue or other debris generated during the aforenoted procedures, the new and improved Yankauer suction tip and tubing aspirator can easily and quickly unclog or unblock the clogged, blocked, or occluded aspirator by quickly discharging any bits of tissue or other debris clogging, blocking, or occluding the aspirator tip without the need to switch or change to a new or fresh alternative or second aspirator.

### SUMMARY OF THE INVENTION

The present invention is directed toward a new and improved Yankauer suction tip and tubing aspirator wherein, in lieu of a single lumen characteristic of conventional Yankauer suction tip and tubing aspirators, and through which aspiration is normally conducted, the new and improved Yankauer suction tip and tubing aspirator comprises dual lumens or a pair of lumens that are integrally connected together in a top-to-bottom or side-by-side array. A first one of the pair of lumens comprises the primary lumen through which air, tissue, and debris is normally aspirated, while the second lumen is provided with a flexible stylus which is axially or longitudinally movable, in a manually-controlled reciprocating manner, within the second lumen such that the forward or distal end portion of the stylus is adapted to move through the open, forward or distal end portion of the second lumen and enter a forward end portion of the first or primary lumen that is located at a position upstream of the open tip portion of the first or primary lumen of the Yankauer suction tip and tubing aspirator through which aspiration is being achieved. Accordingly, if the open tip portion of the Yankauer suction tip and tubing aspirator, that is, the open tip portion of the first or primary lumen of the Yankauer suction tip and tubing aspirator, should become clogged, blocked, or occluded, as a result of tissue or other debris generated during the dental or surgical procedure, then the stylus can be moved through the second lumen such that the forward or distal end portion of the stylus moves into the forward end portion of the first or primary lumen, that is located at a position upstream of the open tip portion of the first or primary lumen of the Yankauer suction tip and tubing aspirator, and continues to move toward and through the open tip portion of the first or primary lumen of the Yankauer suction tip and tubing aspirator, through which aspiration is being achieved, so as to protrude from the open tip portion of the first or primary lumen of the Yankauer suction tip and tubing aspirator and thereby effectively unclog or unblock the clogged, blocked, or occluded first or primary lumen of the Yankauer suction tip and tubing aspirator.

It is to be further noted that the rearward or proximal end of the stylus, disposed within the second lumen of the Yankauer suction tip and tubing aspirator, has a thumb tab integrally connected thereto, and a longitudinally oriented slot is defined within an outer surface wall portion of the second lumen. In this manner, the thumb tab can be utilized to move the stylus in a reciprocating manner through the second lumen of the Yankauer suction tip and tubing aspirator as a result of moving through the longitudinally oriented slot defined within the outer surface wall portion of the second lumen. When, for example, the thumb tab is grasped so as to move the stylus forwardly within the second lumen of the Yankauer suction tip and tubing aspirator, the stylus will be moved in accordance with the mode of operation previously noted, that is, through the second lumen such that the forward or distal end portion of the stylus moves into the forward end portion of the first or primary lumen, that is located at a position upstream of the open tip portion of the first or primary lumen of the Yankauer suction tip and tubing aspirator, through the forward end portion of the first or primary lumen of the Yankauer suction tip and tubing aspirator, and through the open tip portion of the first or primary lumen of the Yankauer suction tip and tubing aspirator so as to protrude from the open tip portion of the first or primary lumen of the Yankauer suction tip and tubing aspirator by means of a predetermined distance so as to effectively unclog or unblock the clogged, blocked, or occluded first or primary lumen of the Yankauer suction tip and tubing aspirator. In accordance with further teachings, principles, and features of this new and improved Yankauer suction tip and tubing aspirator, suitable locking mechanisms can be utilized to secure the stylus at both the retracted and extended or deployed positions.

An alternative embodiment of the new and improved Yankauer suction tip and tubing aspirator comprises the use of a coiled spring, or other elastic device that stores mechanical energy, which is operatively connected to the thumb tab and the stylus. More particularly, the coiled spring, which is disposed within the proximal end of the second lumen so as to operatively engage the proximal end of the stylus, will be compressed by the proximal end of the stylus when the stylus is moved from its extended or deployed position to its retracted position by means of the thumb tab, and a suitable locking mechanism will be employed to maintain the stylus at its retracted position. When the locking mechanism is released, the coiled spring will effectively drive or move the stylus through the second and first lumens, respectively, as has been previously noted. When utilizing this embodiment of the Yankauer suction tip and tubing aspirator, the default position preferably comprises the unlocked position at which the stylus has been moved to its extended or deployed position so as to protrude slightly from the open tip end portion of the first or primary lumen of the Yankauer suction tip and tubing aspirator and thereby, accordingly, as a result of occluding the open tip or end portion of the first or primary lumen, aspiration will effectively be prevented from occurring, as will any noise which normally accompanies aspiration. This is sometimes a preferred condition during various procedures so as to effectively improve communication conditions among surgical personnel. Alternatively, the coiled spring or similar device may be operatively engaged with the stylus such that the default position is the retracted position whereby after the stylus is manually moved to the extended or deployed position, and the thumb tab or knob is effectively released or manually controlled so as to effectively return the stylus to its original retracted position, the coiled spring or the like will inherently bias the stylus back to its retracted position.

### BRIEF DESCRIPTIONS OF THE DRAWINGS

Various other features and attendant advantages of the present invention will be more fully appreciated from the following detailed description when considered in connection with the accompanying drawings in which like reference characters designate like or corresponding parts throughout the several views, and wherein:

FIG. 1 is a perspective view of a conventional or PRIOR ART regular tip vented Yankauer suction tip and tubing aspirator;

FIG. 2 is a perspective view of a conventional or PRIOR ART bulb tip vented Yankauer suction tip and tubing aspirator;

FIG. 3 is a side elevational view of a first embodiment of a new and improved Yankauer suction tip and tubing aspirator as constructed in accordance with the principles and teachings of the present invention and comprising the first and second lumens, the stylus disposed at its extended or deployed position so as to extend outwardly from the distal end opening of the first lumen, and a coiled spring operatively engaged with the stylus such that the default position of the stylus is its extended or deployed position;

FIG. 4 is a side elevational view of the stylus of the present invention which is to be inserted into the second lumen of the new and improved Yankauer suction tip and tubing aspirator as disclosed within FIG. 3;

FIG. 5 is a top plan view of the new and improved Yankauer suction tip and tubing aspirator as illustrated within FIG. 3, showing the thumb tab and its disposition with respect to the longitudinally oriented slot defined within the second lumen so as to permit the stylus to be moved longitudinally within the second lumen between its retracted and extended or deployed positions, the stylus being shown at its retracted position so as to compress the coiled spring disposed within the proximal end of the second lumen;

FIG. 6 is a side elevational view of a second embodiment of a new and improved Yankauer suction tip and tubing aspirator as constructed in accordance with the principles and teachings of the present invention and comprising the first and second lumens, the stylus disposed at its retracted position, and a coiled spring operatively engaged with the stylus such that the default position of the stylus is its retracted position.

### DETAILED DESCRIPTION OF THE ILLUSTRATED EMBODIMENT

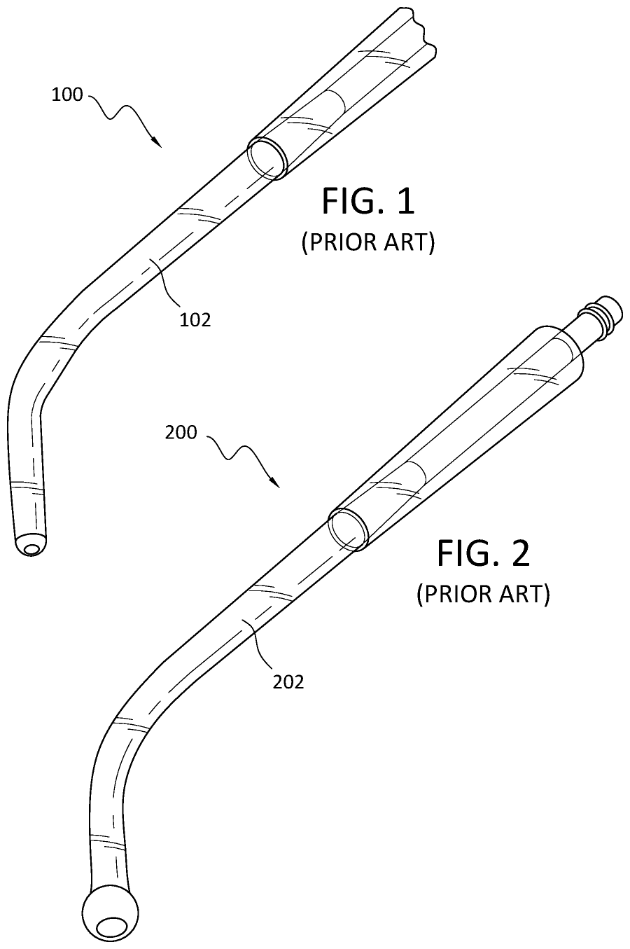

Referring now to the drawings, and initially to FIGS. 1 and 2 thereof, it is seen that a conventional or PRIOR ART regular tip vented Yankauer suction tip and tubing aspirator is disclosed within FIG. 1 and has been generally indicated by means of the reference character 100. More particularly, it is seen that the regular tip vented Yankauer suction tip and tubing aspirator, as disclosed within FIG. 1, comprises a single lumen 102 through which aspiration is achieved. In a similar manner, a conventional or PRIOR ART bulb tip vented Yankauer suction tip and tubing aspirator is disclosed within FIG. 2 and has been generally indicated by means of the reference character 200. More particularly, it is seen that the bulb tip vented Yankauer suction tip and tubing aspirator, as disclosed within FIG. 2, comprises a single lumen 202 through which aspiration is achieved.

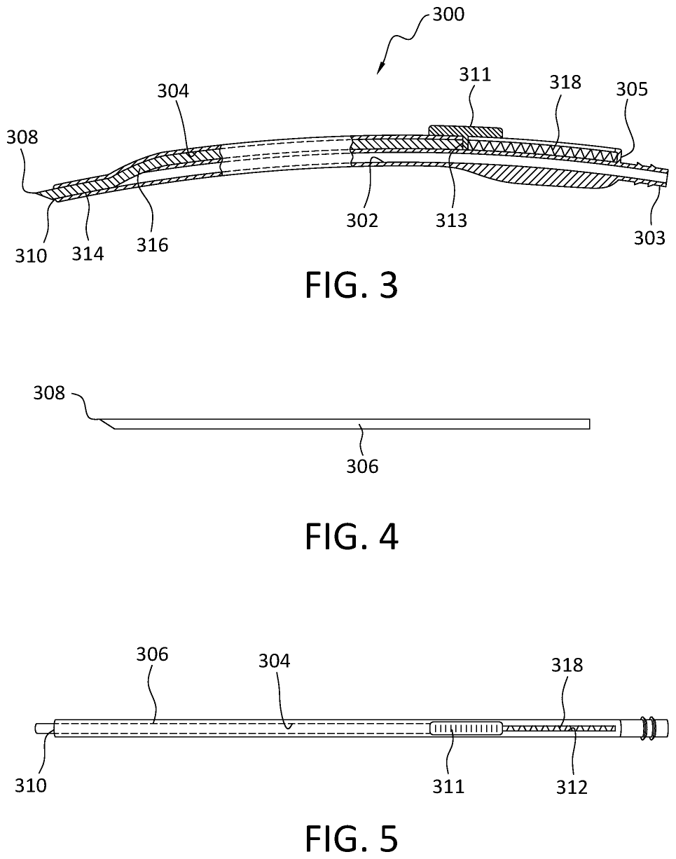

With reference now being made to FIGS. 3-5, it is to be appreciated that the present invention is directed toward a new and improved Yankauer suction tip and tubing aspirator which is disclosed and generally indicated by the reference character 300. More particularly, it is seen that in accordance with the principles and teachings of the present invention, and in lieu of a single lumen, as is characteristic of conventional Yankauer suction tip and tubing aspirators, and through which aspiration is normally conducted, a first embodiment of the new and improved Yankauer suction tip and tubing aspirator 300 is seen to comprise a pair of lumens 302 and 304 that are integrally connected together in a top-to-bottom or side-by-side array as can best be appreciated from FIG. 1. The first one of the pair of lumens 302 comprises the primary lumen through which air, tissue, and debris is normally aspirated as a result of the proximal end 303 of the first lumen 302 being fluidically connected to a conventional suction or aspiration device, not shown, while the second lumen 304, which is closed at its proximal end 305, is provided with a flexible stylus 306, as shown in FIG. 4, which is axially or longitudinally movable, in a manually-controlled or spring-biasing reciprocating manner, within the second lumen 304 such that the forward or distal end portion 308 of the stylus, which effectively has a beveled shape subtending an angle of between 30-45°, is adapted to move through the open, forward or distal end portion of the second lumen 304 and enter a forward end portion of the first or primary lumen 302 that is located at a predetermined position upstream of the open tip portion 310 of the first or primary lumen 302 of the Yankauer suction tip and tubing aspirator 300 through which aspiration is being achieved, such predetermined position or distance being, for example, between 30-40 mm.

It is to be noted that the provision of the beveled forward or distal end portion 308 is provided such that when the stylus 306 moves through the second lumen 304, enters the forward end portion of the first or primary lumen 302, and is adapted to continue to move forward toward the open tip portion 310 of the first or primary lumen 302 of the Yankauer suction tip and tubing aspirator 300, the beveled forward or distal end portion 308 will effectively encounter a side wall portion of the first lumen 302, and due to the flexibility of the stylus, will be caused and/or permitted to bend or flex such that the beveled forward or distal end portion 308 can continue to move through the first lumen 302 and eventually protrude out from the open tip portion 310 of the first or primary lumen 302 of the Yankauer suction tip and tubing aspirator 300 so as to effectively unclog or unblock the clogged, blocked, or occluded tip portion 310 of the first or primary lumen 302 of the Yankauer suction tip and tubing aspirator 300. Accordingly, if the open tip portion 310 of the Yankauer suction tip and tubing aspirator 300, that is, the open tip portion 310 of the first or primary lumen 302 of the Yankauer suction tip and tubing aspirator 300, should become clogged, blocked, or occluded, as a result of tissue or other debris generated during the dental or surgical procedure, then the stylus 306 can be moved through the second lumen 304 such that the beveled forward or distal end portion 308 of the stylus 306 moves into the forward end portion of the first or primary lumen 302, that is located at the aforenoted particular or predetermined position upstream of the open tip portion 310 of the first or primary lumen 302 of the Yankauer suction tip and tubing aspirator 300, and continues to move toward and through the open tip portion 310 of the first or primary lumen 302 of the Yankauer suction tip and tubing aspirator 300, through which aspiration is being achieved, so as to protrude a predetermined distance from the open tip portion 310 of the first or primary lumen 302 of the Yankauer suction tip and tubing aspirator 300 and thereby effectively unclog or unblock the clogged, blocked, or occluded first or primary lumen 302 of the Yankauer suction tip and tubing aspirator 300.

Continuing further, and with reference continuing to be made to FIGS. 3-5, it is to be noted that the rearward or proximal end of the stylus 306, disposed within the second lumen 304 of the Yankauer suction tip and tubing aspirator 300, has a thumb tab, protrusion, knob, or the like 311 integrally connected thereto, and a longitudinally oriented slot 312 is defined within an outer surface wall portion of the second lumen 304. In this manner, the thumb tab or knob 311 can be utilized to move the stylus 306 in a reciprocating manner through the second lumen 304 of the Yankauer suction tip and tubing aspirator 300, as a result of the thumb tab or knob 311 being fixedly connected to the proximal end of the stylus 306 by means of any suitable connection 313 which connects the thumb tab or knob 311 to the stylus 306, wherein the connection or junction 313 defined between the thumb tab or knob 311 moves through the longitudinally oriented slot 312 defined within the outer surface wall portion of the second lumen 304. Accordingly, when, for example, the thumb tab or knob 311 is grasped so as to move the stylus 306 forwardly within the second lumen 304 of the Yankauer suction tip and tubing aspirator 300, the stylus 306 will be moved in accordance with the mode of operation previously noted, that is, through the second lumen 304 such that the forward or distal end portion 308 of the stylus 306 moves into a forward end portion 314 of the first or primary lumen 302, which is located at a position upstream of the open tip portion 310 of the first or primary lumen 302 of the Yankauer suction tip and tubing aspirator 300 and which effectively coincides with a junction 316 of the first and second lumens 302,304, through the forward end portion 314 of the first or primary lumen 302 of the Yankauer suction tip and tubing aspirator 300, and through the open tip portion 310 of the first or primary lumen 302 of the Yankauer suction tip and tubing aspirator 300 so as to protrude from the open tip portion 310 of the first or primary lumen 302 of the Yankauer suction tip and tubing aspirator 300, by means of a predetermined distance, and thereby effectively dislodge any tissue or debris which may be disposed within said open tip portion 310 of the first lumen 302 of the Yankauer suction tip and tubing aspirator 300 so as to effectively unclog or unblock the clogged, blocked, or occluded first or primary lumen 302 of the Yankauer suction tip and tubing aspirator 300. The tissue or debris effectively dislodged from the primary lumen 302 by means of the stylus 306 can then be easily and quickly manually removed, and subsequently, the dental or surgical procedure can quickly and readily resume as a result of the primary lumen 302 now being fully open so as to provide its aspirational functions. In accordance with further teachings, principles, and features of this new and improved Yankauer suction tip and tubing aspirator 300, suitable locking mechanisms, not shown, can be utilized to secure the stylus 306 at both its retracted and extended or deployed positions. In addition, it is to be noted that the forward or distal end portion 308 of the stylus can have various configurations, such as, for example, the forward or distal end portion 308 can be beveled, flat, blunt, concave, convex, and the like.

With reference being made specifically to FIG. 3 for clarity purposes, an alternative embodiment of the new and improved Yankauer suction tip and tubing aspirator 300 comprises the use of a coiled spring 318 which is operatively connected to the thumb tab or knob 311 and the stylus 306. More particularly, the coiled spring 318, which is disposed within the proximal end of the second lumen 304 such that the distal end of the spring 318 is operatively engaged with the proximal end of the stylus 306 while the proximal end of the spring abuts an end wall portion of the second lumen 304, will be compressed by the proximal end of the stylus 306 when the stylus 306 is moved from its extended or deployed position to its retracted position by means of the thumb tab or knob 311, and a suitable locking mechanism, not shown, will be employed to maintain the stylus 306 at its retracted position. When the locking mechanism is released, the coiled spring 318 will effectively drive or move the stylus 306 through the second and first lumens 302,304, respectively, as has been previously noted. When utilizing this embodiment of the Yankauer suction tip and tubing aspirator 300, the default position may preferably comprise the unlocked position at which the coiled spring 318 or the like is disposed in its expanded state whereby stylus 306 has been moved to its extended or deployed position so as to protrude slightly from the open tip end portion 310 of the first or primary lumen 302 of the Yankauer suction tip and tubing aspirator 300. Accordingly, as a result of occluding the open tip or end portion 310 of the first or primary lumen 302, aspiration will effectively be prevented from occurring, as will any noise which normally accompanies aspiration procedures. This is sometimes a preferred condition during various procedures so as to effectively improve communication conditions among surgical personnel.

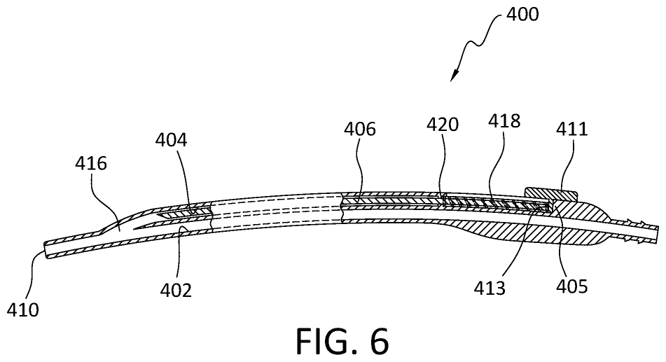

To the contrary, the default position may be that wherein the coiled spring is likewise disposed in its expanded state, however, the stylus has been moved to its retracted position such that the primary lumen is already open whereby aspiration may be immediately performed as or when necessary, and yet the stylus is readied to be moved to its extended or deployed position so as to unclog or unblock the clogged, blocked, or occluded primary lumen when necessary. With reference therefore being made to FIG. 6, a second embodiment of a new and improved Yankauer suction tip and tubing aspirator has been developed to achieve this mode of operation, and is disclosed and generally indicated by the reference character 400. It is to be noted that component parts of this second embodiment of a new and improved Yankauer suction tip and tubing aspirator 400, which correspond to component parts of the first of a new and improved Yankauer suction tip and tubing aspirator 300, will be provided with corresponding reference numbers except that they will be within the 400 series, and therefore no detailed discussion of the same is necessary and will not be provided.

More particularly, with reference being made to FIG. 6, it is seen that a coiled spring 418 or the like could be located downstream of the thumb tab or knob 411, or upon the opposite side from where the coiled spring 311 or the like was located relative to the thumb tab or knob 311 in the first embodiment of the new and improved Yankauer suction tip and tubing aspirator 300 illustrated within FIGS. 3-5. It is also seen that the spring 418 annularly surrounds a proximal end portion of the stylus 406, that the distal end of the spring 418 or the like abuts an annular wall portion of the second lumen 404 which extends radially inwardly as disclosed at 420, while the proximal end of the spring 418 or the like abuts the connection 413 defined between the thumb tab or knob 411 and the stylus 406. Consequently, it can be appreciated that when the thumb tab or knob 411 is grasped so as to move the stylus 406 from its retracted position to its extended or deployed position so as to remove tissue or debris which may be clogging or blocking the open end 410 of the first or primary lumen 402, the connection 413, defined between the thumb tab or knob 411 and the stylus 406, will effectively compress the coiled spring or the like 418 since the distal end of the coiled spring or the like 418 abuts against the annular wall portion 420 disposed internally within the second lumen 404. Subsequently, once the clogging, blockage, or occlusion of the first or primary lumen 402 has been dislodged, unclogged or unblocked, the coiled spring or the like 418 can be permitted to expand and thereby move the stylus 406 back to its original position as illustrated within FIG. 6 which defines its default position.

Obviously, many variations and modifications of the present invention are possible in light of the above teachings. It is therefore to be understood that within the scope of the appended claims, the present invention may be practiced otherwise than as specifically described herein.

What is claimed as new and desired to be protected by Letters Patent, is:

1. A suction tip and tubing aspirator, comprising:
a first lumen having a first, proximal, open end portion adapted to be operatively connected to a source of suction, and a second, distal, open end portion adapted to be inserted into a site from which tissue or debris is to be aspirationally removed;
a second lumen integrally connected to said first lumen; and
a stylus movably disposed within said second lumen between retracted and extended or deployed positions such that when said stylus is moved from said retracted position to said extended or deployed position, a distal end portion of said stylus will protrude outwardly from said second, distal, open end portion of said first lumen so as to dislodge any tissue or debris which has clogged, blocked, or occluded said second, distal, open end portion of said first lumen.

2. The suction tip and tubing aspirator as set forth in claim 1, wherein:
said first and second lumens are disposed in one of a top-to-bottom and side-by-side array.

3. The suction tip and tubing aspirator as set forth in claim 1, wherein:
said stylus is fabricated from a suitable flexible material.

4. The suction tip and tubing aspirator as set forth in claim 3, wherein:
said distal end portion of said stylus has a beveled configuration such that when said stylus is moved through said second lumen and said beveled distal end portion of said stylus moves into said first lumen, said beveled second end portion of said stylus will encounter a side wall portion of said first lumen and will cause said flexible stylus to bend or flex so as to permit said second end portion of said stylus to continue to move through said first lumen, and protrude outwardly from said second open end portion of said first lumen so as to dislodge any tissue or debris clogging, blocking, or occluding said second open end portion of said first lumen.

5. The suction tip and tubing aspirator as set forth in claim 1, further comprising:
a thumb tab or knob fixedly connected to said stylus by a suitable connection so as to move said stylus between said retracted and extended or deployed positions.

6. The suction tip and tubing aspirator as set forth in claim 5, further comprising:
a longitudinal slot defined within a side wall portion of said second lumen so as to permit said suitable connection, defined between said thumb tab or knob and said stylus, to move through said longitudinal slot and to move said stylus between said retracted and extended or deployed positions as controlled by said thumb tab or knob.

7. The suction tip and tubing aspirator as set forth in claim 5, further comprising:
a coiled spring operatively engaged with a proximal end portion of said stylus so as to normally bias said stylus toward said extended or deployed position.

8. The suction tip and tubing aspirator as set forth in claim 5, further comprising:
a coiled spring operatively engaged with a proximal end of said stylus such that when said stylus is moved to its retracted position, said proximal end of said stylus will compress said coiled spring whereby said coiled spring will tend to bias said stylus toward said extended or deployed position.

9. The suction tip and tubing aspirator as set forth in claim 5, further comprising:
a coiled spring operatively engaged with a proximal end portion of said stylus so as to normally bias said stylus toward said retracted position.

10. The suction tip and tubing aspirator as set forth in claim 5, further comprising:
a coiled spring operatively engaged with a proximal end of said stylus such that when said stylus is moved to its extended position, said proximal end of said stylus will compress said coiled spring whereby said coiled spring will tend to bias said stylus back toward said retracted position.

11. The suction tip and tubing aspirator as set forth in claim 5, further comprising:
a coiled spring operatively engaged with a proximal end portion of said stylus and disposed upon a proximal side of said connection, defined between said thumb tab or knob and said proximal end portion of said stylus, such that when said stylus is moved to its retracted position, said proximal end portion of said stylus will compress said coiled spring whereby said coiled spring will tend to bias said stylus toward said extended or deployed position, whereas when said coiled spring is operatively engaged with a proximal end portion of said stylus and disposed upon a distal side of said connection, defined between said thumb tab or knob and said proximal end portion of said stylus, such that when said stylus is moved to its extended position, said connection will compress said coiled spring whereby said coiled spring will normally bias said stylus back toward said retracted position.
